(19) United States
(12) Patent Application Publication
(10) Pub. No.: US 2017/0143046 A1
(43) Pub. Date: May 25, 2017

(54) A BIB WITH POUCH

(71) Applicant: FOR MY BABY LIMITED, Lower Hutt (NL)

(72) Inventor: MARIA CHRISTINA GIANNAKAKIS, Wellington (NZ)

(73) Assignee: FOR MY BABY LIMITED, Lower Hutt (NZ)

(21) Appl. No.: 15/325,917

(22) PCT Filed: Sep. 3, 2014
(86) PCT No.: PCT/NZ14/00188

§ 371 Date: Jan. 12, 2017

(51) Int. Cl. A41B 13/10; A61F 5/37; A41B 13/06

(52) U.S. Cl. CPC A41B 13/103; A41B 13/06; A61F 5/3723

(57) ABSTRACT

The invention relates to a bib useful for securing a wearer's hands and/or arms, the bib comprising a first outer-facing panel, a second body-facing panel, first securing means provided in or adjacent the second body-facing panel and adapted in use to secure the hands and/or arms of a wearer, and second securing means adapted in use to secure the bib to the wearer, the arrangement being such that in use the wearer's hands and/or arms are secured substantially within or by the first securing means at the front of the wearer's body.

### FIELD OF THE INVENTION

The present invention relates to a bib with a pouch. In particular, the invention relates to a bib with a pouch useful for restraining an infant's hands and/or arms, for example while undertaking a nappy (diaper) change.

### BACKGROUND OF THE INVENTION

Babies and infants usually wear nappies or diapers (hereafter diapers), until they are toilet-trained. As babies grow, they become more aware of their surroundings, more active and more independent. Changing the diaper of a new baby is relatively easy, but changing the diaper of an older infant can be more problematic, as the infant is active, moves about much more than a new baby and may resist having its diaper changed.

When changing a diaper, it is often more difficult to control the infant's arms than its legs and lower body. The infant will often wave its arms around, and try to grab hold of nearby items. It may also try to interfere with the diaper changing process using its hands. While this may be a minor problem, but it can be more concerning, for example if the infant's hands are able to touch the contents of a dirty diaper.

Parents and caregivers encountering this type of problem during a diaper change often desire to restrain an infant's arms and/or hands, and sometimes resort to ad hoc means to do this. This is not always successful and in some case may not be safe and/or hygienic.

It is also sometimes desirable to restrain and infant's arms and/or hands in other circumstances, for example during feeding or during administration of medicines.

A need therefore exists to provide a safe, cost effective and practical means of restraining an infant's hands and/or arms.

### SUMMARY OF THE INVENTION

In a first aspect, the present invention provides a bib comprising:
a first outer-facing panel,
a second body-facing panel,
first securing means provided in or adjacent the second body-facing panel and adapted in use to secure the hands and/or arms of a wearer, and
second securing means adapted in use to secure the bib to the wearer, the arrangement being such that in use the wearer's hands and/or arms are secured substantially within or by the first securing means at the front of the wearer's body.

Preferably, the wearer's hands and/or arms have a range of movement at the front of its body.

Preferably, only the second securing means or a portion of the second securing means extends behind the wearer's body.

Preferably, the first securing means is a pouch provided in or adjacent the second body-facing panel.

Preferably, the pouch is formed in the body-facing panel.

Preferably, the pouch is formed separately from and is adjacent to the body-facing panel.

Preferably, the entrance to the pouch is expandable.

Preferably, the pouch extends, in use, substantially up to or adjacent the wearer's arm pits.

In a second aspect, the present invention comprises a method of restraining an infant's hands and/or arms, the method comprising:
providing a bib comprising a first outer-facing panel, a second body-facing panel, first securing means provided in or adjacent the second body-facing panel and adapted in use to secure the hands and/or arms of a wearer, and second securing means adapted in use to secure the bib to the wearer, and
locating the wearer's hands and/or arms substantially within the first securing means such that the wearer's hands and/or arms are secured substantially within or by the first securing means at the front of the wearer's body.

This brief summary of the invention broadly describes the features and advantages of certain embodiments of the invention. Further features and advantages will be described in the detailed description of the invention that follows.

Novel features that are believed to be characteristic of the invention will be better understood from this detailed description when considered in connection with the accompanying drawings. However, the accompanying drawings are intended to help illustrate the invention or assist with understanding the invention, and are not intended to define the scope of the invention.

### BRIEF DESCRIPTION OF THE DRAWINGS

FIG. 1: shows a front outer-facing view of a bib with a pouch, according to one aspect of the present invention.

FIG. 2: shows a rear body-facing view of the bib shown in FIG. 1.

FIG. 3: shows a perspective view of the bib of the present invention, in use.

FIG. 4: shows a further perspective view of the bib of the present invention, in use.

FIG. 5: shows a still further perspective view of the bib of the present invention, in use.

### DETAILED DESCRIPTION OF THE INVENTION

The present invention provides a bib with a pouch. The bib of the present invention is adapted for restraining an infant's hands and/or arms and in particular an infant's hands when desired, for example while undertaking a diaper change.

In broad terms, the bib of the present invention comprises a front side comprising an outer-facing panel and a rear side comprising a body-facing panel, the rear side being provided with a pouch. The pouch is sufficiently large that it can accommodate and secure the hands and/or arms of an infant wearing the bib. The bib also comprises the features of a regular bib, such as a means for securing the bib about the neck of an infant.

A preferred embodiment of the bib of the present invention is shown in the accompanying drawings.

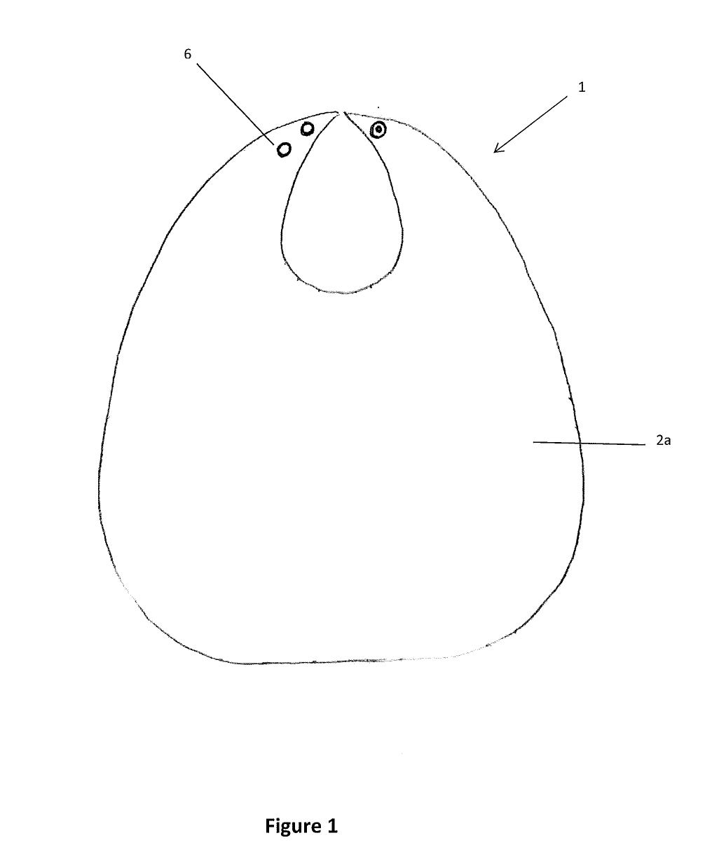

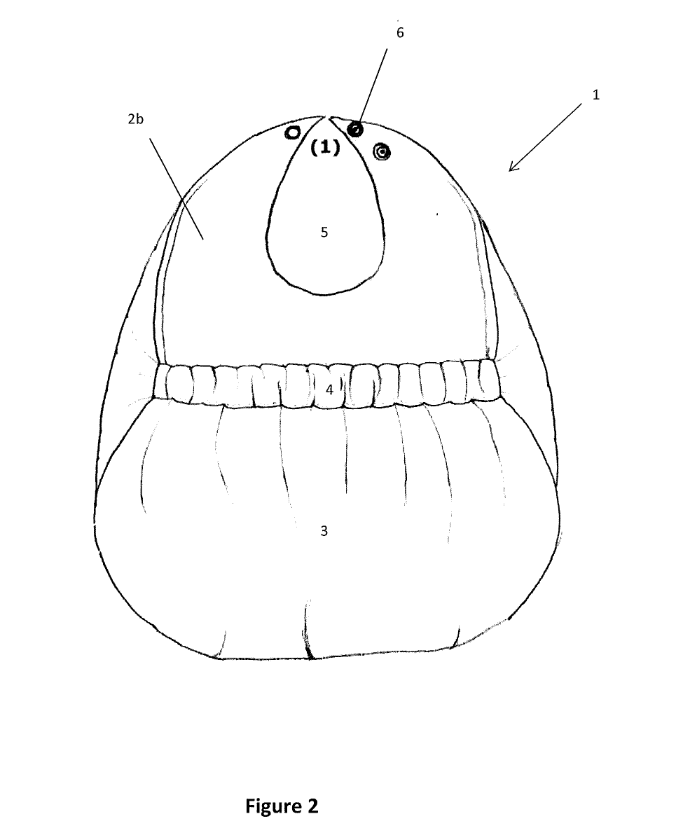

As shown in FIGS. 1 and 2, the bib 1 comprises a front outer-facing panel 2a and a rear-body facing panel 2b. A first securing means, such as a pouch 3, is provided as part of or adjacent the rear body-facing panel 2b of the bib 1. The first securing means 3 has an opening such as entrance portion 4.

This bib of the present invention may be made of any suitable flexible material, such as a woven or non-woven fabric. In one form of the invention, the bib 1 is preferably formed of several layers of a suitable fabric, sewn together.

Fabrics of contrasting colours may be used so that, for example, a front outer-facing panel 2a and a rear body-facing panel 2b may be of different colours.

It should be appreciated that the bib 1 may be formed of other materials, such as a suitable plastics material.

The first securing means provided adjacent to or forming part of the body-facing panel 2b is preferably a pouch 3. The pouch 3 may be formed in the body-facing panel 2b itself, or may be a separate panel that is attached to the body-facing panel 2b and/or the outer-facing panel 2a, as shown in FIG. 2.

Preferably, the entrance portion 4 is expandable, making it easier to insert an infant's hands and/or arms into the pouch 3, as further described below. This aspect of the invention may be achieved, for example, by providing an elasticated entrance portion 4 at the top of the pouch 3.

Although the bib shown in the accompanying drawings has a single pouch 3 provided in the body-facing panel, other arrangements may be provided. For example, several pouches or other restraining means such as ties may be provided.

There is also a cut out portion 5 to accommodate the neck of the infant and second securing means 6 to secure the bib 1 about the neck of the infant. As shown in FIGS. 1 and 2, the second securing means is a snap lock fastener, although it will be appreciated that other securing means such as elongate ties may also be provided. Several snap lock fasteners may be provided so that different neck sizes may be accommodated.

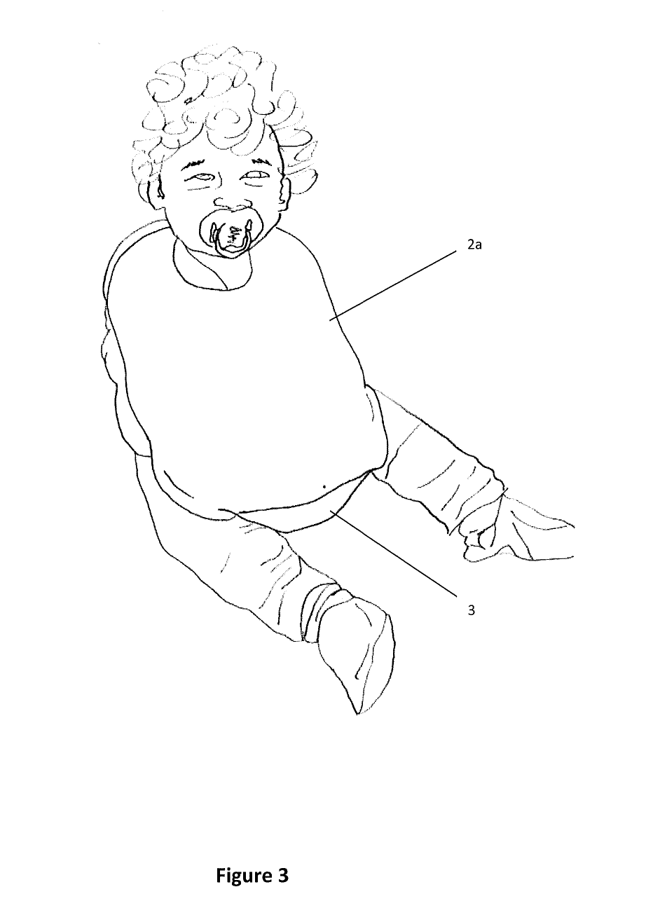

Referring now to FIGS. 3 to 5, in use, the bib 1 is secured about the neck of an infant. The bib 1 may be used in the usual way, that is, as a regular bib. When it is desired or necessary to restrain the infant's arms and/or hands, for example when the infant's diaper needs to be changed, the infant's hands and arms are moved into the pouch 3. This is achieved by stretching or otherwise expanding the expandable entrance portion 4 of the pouch 3.

Once the infant's hands and arms are inserted into the pouch 3, the expandable portion 4 is released by the caregiver. This results in the pouch tightening up somewhat around the infant's hands and/or arms, so that they are securely retained within the pouch 3.

As can be seen from FIGS. 3 to 5, the pouch 3 is of sufficient size and dimension to comfortably accommodate the infant's hands and arms, so that the infant does not feel overly restricted or retrained. At the same time, however, the infant's hands and/or arms are securely retained within the pouch 3.

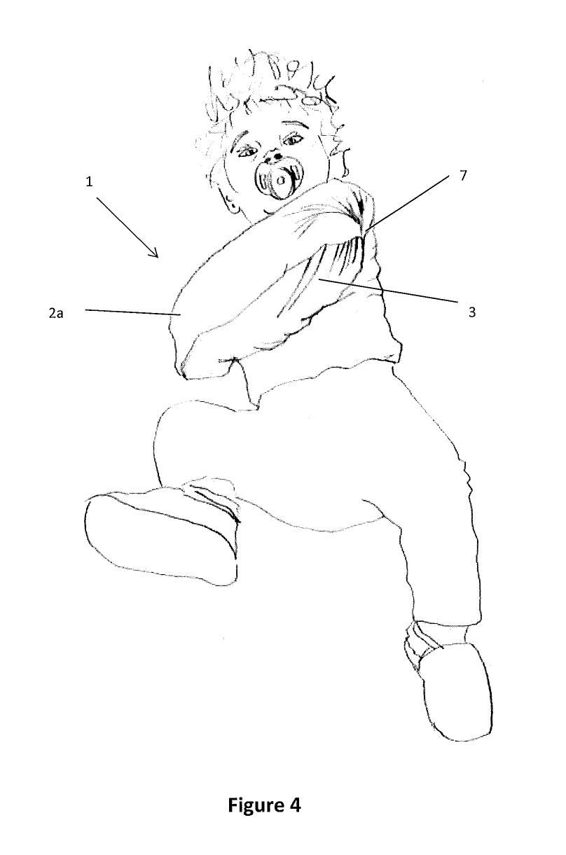

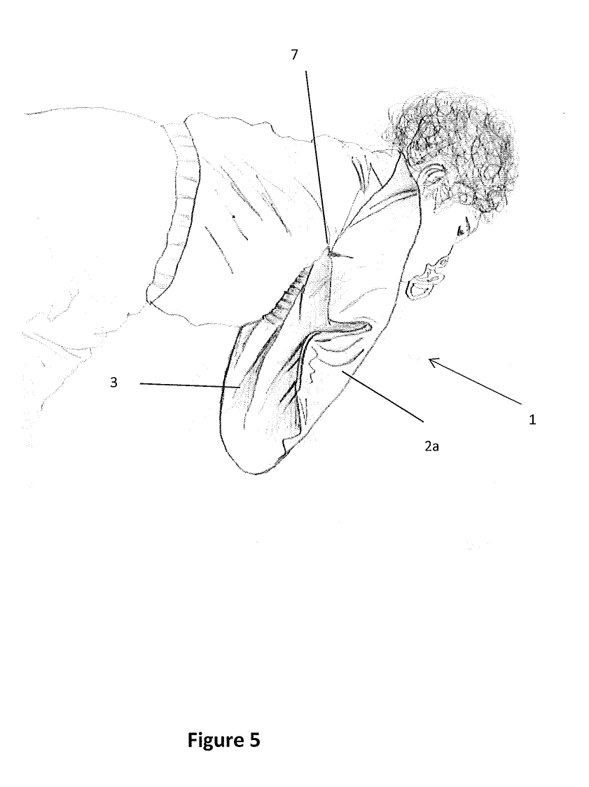

In the embodiment shown in the accompanying drawings, this is achieved by providing a bib 1 and pouch 3 of a size and dimension such that the bib 1 extends from the infant's neck down to underneath the infant's arm pits (at 7 in FIGS. 4 and 5).

One advantage of the bib of the present invention is that, apart from the neck portion of the bib (which comprises the means for securing the bib to the infant) the bib does not extend around the back of the infant. This means that, although the infant's hands and/or arms are retained out of the way of a diaper changing process, the infant still has a range of movement at the front of its body. For example, the infant will have some side to side movement. This may be compared with other arrangements or devices that extend around the infant's back and more severely restrict or restrain the infant's movement, possible causing distress to the infant.

The present invention and its embodiments have been described in detail. However, the scope of the present invention is not intended to be limited to the embodiment described in the specification. Modifications and variations may be made to the disclosed embodiment without departing from the scope or essential characteristics of the present invention.

What is claimed is:

1. A bib comprising:
a first outer-facing panel,
a second body-facing panel,
first securing means provided in or adjacent the second body-facing panel and adapted in use to secure the hands and/or arms of a wearer, and
second securing means adapted in use to secure the bib to the wearer,
wherein the first securing means comprises a pouch, wherein the pouch is provided in or adjacent to the body-facing panel, and wherein the pouch comprises an expandable entrance portion that transverses the body-facing panel and that expands away from the body-facing panel,
the arrangement being such that in use the wearer's hands and/or arms are secured substantially within or by the first securing means at the front of the wearer's body.

2. The bib as claimed in claim 1, wherein, in use, the wearer's hands and/or arms have a range of movement in the pouch.

3. The bib as claimed in claim 1 or claim 2, wherein only the second securing means or a portion of the second securing means extends behind the wearer's body.

4. The bib as claimed in claim 1, wherein the pouch is adjacent to the second body-facing panel.

5. The bib as claimed in claim 1, wherein the pouch is formed in the body-facing panel.

6. The bib as claimed in claim 1, wherein the pouch is formed separately from and is adjacent to the body-facing panel.

7. The bib as claimed in claim 1, wherein the expandable entrance portion of the pouch has an expandable entrance portion is elasticated.

8. The bib as claimed in claim 1, wherein the pouch extends, in use, substantially up to or adjacent the wearer's arm pits.

9. A method of restraining an infant's hands and/or arms, the method comprising:
providing a bib comprising a first outer-facing panel, a second body-facing panel, first securing means adapted in use to secure the hands and/or arms of a wearer, and second securing means adapted in use to secure the bib to the wearer,
wherein the first securing means comprises a pouch, wherein the pouch is provided in or adjacent to the body-facing panel, and wherein the pouch comprises an expandable entrance portion that transverses the body-facing panel and that expands away from the body-facing panel, and
locating the wearer's hands and/or arms substantially within the first securing means such that the wearer's hands and/or arms are secured substantially at the front of the wearer's body.
